(19) United States
(12) Patent Application Publication
(10) Pub. No.: US 2024/0226893 A1
(43) Pub. Date: Jul. 11, 2024

(54) Device and Method for Sorting Biological Cells

(71) Applicants: IMEC VZW, Leuven (BE); Janssen Biotech Inc., Horsham, PA (US)

(72) Inventors: Peter Peumans, Herfelingen (BE); Olivier Henry, Kraainem (BE); Willem Van Roy, Bierbeek (BE); Tim Stakenborg, Heverlee (BE); Alexander Graham Irwin Tulloch, Malvern, PA (US)

(21) Appl. No.: 18/559,029

(22) PCT Filed: May 12, 2022
(86) PCT No.: PCT/US2022/029026

§ 371 Date: Nov. 3, 2023

Related U.S. Application Data
(60) Provisional application No. 63/188,314, filed on May 13, 2021.

(51) Int. Cl. B01L 3/00

(52) U.S. Cl. CPC B01L 3/502761; B01L 3/502715; B01L 2200/0652; B01L 2300/0654; B01L 2300/0816; B01L 2300/0864; B01L 2300/161; B01L 2400/086; B01L 2400/088

(57) ABSTRACT

A device for sorting biological cells comprises a flow channel configured to pass a flow of liquid carrying the biological cells. The flow channel comprises at least one zone associated with a cell category. Each zone of the flow channel comprises at least one surface coated with molecules having an affinity to the cell category associated with the zone, the at least one surface of the zone being configured to modify, by the molecules of the surface, a movement of a cell belonging to the associated cell category of the zone as the cell passes the zone. The device comprises a detector configured to detect a cell exhibiting at least one modified movement in one zone of the flow channel, and to transmit a trigger signal based on the detection of the cell. The device further comprises an actuator configured to divert the detected cell based on the trigger signal.

### RELATED APPLICATIONS

This application claims the benefit of priority under 35 U.S.C. § 119(e) of U.S. Provisional Application No. 63/188,314, filed May 13, 2021, the content of which is incorporated herein by reference in its entirety.

### FIELD OF THE DISCLOSURE

The present disclosure relates to a device and a method for sorting biological cells.

### BACKGROUND

Sorting of biological cells is important both for cell analysis and cell therapy. According to some sorting methods, sorting is facilitated by labeling the cells. In magnetic-activated cell sorting (MACS), cells are labeled by attaching magnetic markers, while in fluorescent-activated cell sorting (FACS), cells are labeled by attaching fluorescent markers. Other sorting methods use label-free sorting, wherein no markers are attached to the cells.

US2011097793A1 describes a label-free cell sorting device. The device comprises a flow path along which cells flow in a saline solution. The flow path comprises adsorbing regions in the form of strips disposed in an asymmetric fashion to the flow path direction. Due to the adsorbing regions, an adsorbing force acts on target cells, the adsorption force having a constituent perpendicular to the flow path direction. As a result, target cells are separated from non-target constituents.

### SUMMARY

An aspect of the disclosure facilitates sorting of biological cells. A further aspect facilitates label-free sorting of biological cells. Yet a further aspect facilitates the manufacture of cell sorting devices that are cost-effective, have high cell throughput, are compact, and/or are reprogrammable. These and other aspects will be elucidated from the examples described herein. As used herein, and unless otherwise indicated, the term cell refers to a biological cell.

In a first aspect, a device for sorting biological cells comprises a flow channel. The flow channel is configured to facilitate the flow of liquid that comprises cells to be sorted. The flow channel comprises one or more zones, and each zone is associated with a cell category. In this regard, in an example, each zone comprises at least one surface that is coated with molecules that have an affinity to cells belonging to the cell category that is associated with the zone. The molecules modify/influence the movement of these cells as they pass through the zone. Such coated surfaces are referred to herein as a cell movement modifying surfaces.

The device further comprises a detector and an actuator. The detector is configured to sense a cell exhibiting at least one modified movement in one zone of the flow channel, and to transmit a trigger signal based on the detection of the cell exhibiting at least one modified movement. The actuator is configured to divert, based on the trigger signal, the detected cell within the flow of liquid in the flow channel.

Cells may be characterized by the molecules they comprise, e.g., the molecules comprised in the cell membrane. Molecules comprised in the cell membrane may, e.g., be antigens. One way to determine whether a cell comprises a certain cell membrane molecule, or, for example, a cell transmembrane molecule, is to allow the cell to react with a molecule that binds specifically to the cell membrane or transmembrane molecule. For example, an antigen may correspond with one or more antibodies in that the antigen and antibody bind specifically to each other. Consequently, cells may be divided into cell categories depending on which cell membrane molecules they comprise or which molecules the cells bind to. For example, the antigen CD4 (cluster of differentiation 4) molecule in cell membranes is a glycoprotein, which binds to anti-CD4 antibodies. Cells that bind to anti-CD4 antibodies may form one cell category. Such cells may be detected based on their modified movements when they travel through a zone with a cell movement modifying (CMM) surface coated by anti-CD4 antibodies. Analogously, cells that bind to anti-CD8 (cluster of differentiation 8) antibodies may form another cell category. Such cells may be detected based on their modified movements when they travel through a zone with a CMM surface coated by anti-CD8 antibodies. Thus, a cell category may be a category of cells that binds to a specific molecule, such as an antibody.

Within examples, molecules coating at least one CMM surface of the at least one zone of the flow channel comprise one or more antibodies, aptamers, and/or lectins. Antibodies, or aptamers, or lectins may have an affinity to specific cell categories but not to other cell categories. Antibodies, or aptamers, or lectins may bind to specific cell categories but not to other cell categories. Antibodies or aptamers may have a high sensitivity in binding to a cell category. Thus, the percentage of true positive binding events may be high. Antibodies or aptamers may have a high specificity in binding to a cell category. Thus, the percentage of true negative binding events may be high. Alternatively, the molecules coating at least one CMM surface of the at least one zone of the flow channel may comprise other molecules than antibodies, aptamers, and lectins, e.g., comprise other molecules that bind specifically to a cell membrane molecule.

An example of a CMM surface is configured to modify the movement of the cell belonging to the associated cell category of the zone by modifying the speed, direction, and/or path of cells comprised in the cell category. For instance, in an example, the CMM surface of at least one zone of the flow channel is configured to modify the movement of the cell belonging to the associated cell category of the zone by binding and releasing the cell belonging to the associated cell category of the zone. Such a movement may be termed a bind-release movement. Thus, in an example, the CMM surface is configured to bind a cell for a finite amount of time. Binding and releasing the cell may temporarily halt the cell along its travel. In an example, such a modified motion in the form of a modified speed is detectable and the cell can be diverted by the actuator. The cell may not necessarily bind and release at the same point of the CMM surface. For instance, in an example, the CMM surface is configured to let a cell bind to the CMM surface, roll a distance along the CMM surface, and then release from the CMM surface in a bind-roll-release movement. The cell may, e.g., roll along a surface in a direction different from the direction of the flow of liquid. Thus, in some examples, the direction or path of a cell belonging to a category associated with the CMM surface is different from the path of a cell belonging to a category not associated with the CMM surface. Such a modified motion in the form of a modified direction or path is, in an example, detectable, and the cell can be diverted by the actuator upon detection.

In an example, the detector is configured to detect one modified movement of a cell in a zone, e.g., one bind and release event, a halt of motion, a change of speed, a change of direction, or a change of path, and thereby identify the cell as belonging to the cell category associated with the zone and generate the trigger signal. In another example, the detector is configured to detect a threshold number of modified movements of a cell in a zone, e.g., the cell displaying at least 2, at least 5, or at least 10 bind and release events within the zone, and, thereby, identify the cell as belonging to the cell category associated with the zone and generate the trigger signal.

In an aspect, the device facilitates cell sorting. In particular, the device facilitates label-free cell sorting as the cells do not need to be labeled. Cells belonging to a certain category may be detected, e.g., identified, based on whether they display a modified movement, and subsequently diverted. Label-free cell sorting is faster than labeled sorting because in labeled sorting, labels oftentimes need to be removed from the cells after sorting and before further analysis or further processing of the cells can be performed. For instance, in some cases, removal of labels is performed in a washing process which can be costly, bulky, and damaging to the cells. As used herein, the term cell sorting corresponds to the separating of cells belonging to a cell category from cells not belonging to the cell category. Cells that are intended to be sorted out, separated from the other cells are hereinafter referred to as target cells.

It is noted that some devices for label-free cell sorting operate by passively sorting the cells. FIG. 1 illustrates a schematic view of such a device. The device comprises a flow channel with a flow direction, the flow channel having a length in the flow direction, and a width transverse to the flow direction. Cells are injected at an inlet of the flow channel, and during their travel through the flow channel, target cells interact with adsorbing regions 92 comprising antibodies, while non-target cells do not interact. Due to the adsorbing regions 92, an adsorbing force acts on target cells, the adsorption force having a constituent perpendicular to the flow path direction. Consequently, the target cells are diverted by the interaction with the adsorbing regions 92, from the flow path of the non-target cells. Thus, target cells end up at a first outlet 19′ while non-target cells end up at a second outlet 19″.

A device according to the first aspect facilitates active label-free cell sorting. This, in turn, facilitates the manufacture of high cell throughput label-free cell sorting devices that are accurate, compact, cost-effective, and reprogrammable. By detecting a modified movement at a CMM surface, a target cell may be identified and then actively diverted by the actuator.

Aspects disclosed herein facilitate the manufacture of accurate cell sorting devices. A modified movement may be a revealing sign which facilitates determining the cell category of a cell with high accuracy. It may, e.g., be very improbable that a cell exhibits a modified movement, e.g., a bind-release movement or a bind-roll-release movement at a CMM surface of a zone without having an affinity to the molecules coating the CMM surface. Thus, the probability of false-positive detection events may be low. Further, an actuator may deterministically divert a cell. Thus, a high percentage of detected cells may be diverted to the correct outlet. In contrast, a passive device may rely on the stochastic diversion of cells based on multiple stochastic interactions with adsorbing regions.

Aspects disclosed herein facilitate the manufacture of compact cell sorting devices as no CMM surface or any other surface needs to be adapted to divert the target cells to any specific position within the flow channel. For a passive device, such as the device illustrated in FIG. 1, the length and the width may need to be large in order to provide a long enough interaction length to sufficiently divert a target cell from non-target cells. In contrast, the flow channel of a device disclosed herein may be shorter and narrower as a long interaction length may be irrelevant. In principle, one or a few modified movements may be all that is needed to detect a cell such that it can be diverted by the actuator.

FIG. 2 illustrates a compact device 1, wherein target cells, being cells 4′ of a first category, and non-target cells move through a first zone 12′. The first zone 12′ comprises a CMM surface 14, in this case, a surface of a sidewall of the flow channel 10 coated with CMM molecules, to which the target cells 4′ briefly stick in a bind-release movement. The actuator 40 may then divert the target cells 4′ within the flow of liquid in the flow channel such that they end up at a first outlet 19′. Non-target cells may end up at a third outlet 19′″. An actuator 40 may be more effective in diverting cells within a flow channel than the interaction between cells and adsorbing surfaces.

Aspects disclosed herein facilitate the manufacture of cell sorting devices with a high cell throughput as several cells may move in parallel and be detected in parallel. FIG. 3 illustrates a high-throughput device 1, wherein target cells 4′ and non-target cells move through a first zone 12′. As illustrated in FIG. 3, the detector may be configured to detect modified movements of two or more cells 4′ simultaneously as they move across a CMM surface 14 of the flow channel. Thus, compared to a passive device, the cell throughput may be higher for a given device area.

Aspects disclosed herein facilitate the manufacture of cost-effective cell sorting devices. This may be a consequence of the device being compact and/or having a high cell throughput. Less material may thereby be needed to manufacture the device.

Aspects disclosed herein facilitate the manufacture of reprogrammable cell sorting devices. In an active cell sorting device, it may be possible to change which type of modified movement should generate the trigger signal and/or how many modified movements should be needed to generate the trigger signal depending on circumstances. For example, if a higher sensitivity and/or specificity in the sorting is needed, a higher threshold number of modified movements of a cell in a zone may be required to generate the trigger signal. Further, in an active cell sorting device, it may be possible to change which outlet the target cells should be diverted to. In one application, cells 4′ of a first category may be diverted to end up at a first outlet 19′ of a device 1. For another application, the same device 1 may be reprogrammed such that cells 4′ of a first category are diverted to end up at a third outlet 19′″. In contrast, a passive cell sorting device may not be reprogrammable.

An example of the device sorts cells in-vitro. The device may, e.g., be used for sorting cells to be used in cell therapy. In cell therapy, large amounts of cells may need to be introduced into a patient in order to boost the immune system of the patient. In an example, one step in generating the immune-system-boosting cells involves performing cell sorting, and in an example, label-free cell sorting. For example, in CAR-T cell therapy, T-cells are derived from a blood sample by sorting out T-cells from other peripheral blood mononuclear cells (PBMCs) such as monocytes, lymphocytes, B cells, and NK cells. The T-cells may subsequently be genetically engineered to express new chimeric antigen receptors (CAR) that will boost the patient's immune system. A cell sorting device with high cell throughput facilitates a reduction in the processing time of the blood sample and thereby the survival rate of the cells.

An example of the actuator is configured to divert the detected cell within the flow of liquid in the flow channel by:
creating an electric field within the flow of liquid;
creating a jet flow within the flow of liquid by heating the liquid; and/or
creating a surface acoustic wave along an inner surface of the flow channel.

The creation of an electric field within the flow of liquid facilitates diverting the detected cell through dielectrophoresis. In an example, the electric field is applied transverse to the flow direction of the flow of liquid. Examples of the electric field are in the range of 0.1 to 10 MV/m and alternate at frequencies ranging from 1 KHz to 1 MHz.

Creating a jet flow within the flow of liquid by heating the liquid facilitates the diversion of the detected cell using a heater, e.g., a resistive heater.

Creating a surface acoustic wave (SAW) along an inner surface of the flow channel facilitates subjecting the detected cell to an acoustic radiation force that diverts the cell. In an example, the actuator is configured to provide the SAW with a propagation direction transverse to the flow direction of the flow of liquid. An example of the actuator comprises an interdigitated transducer. An example of the interdigitated transducer comprises interdigitated electrodes on a piezoelectric substrate. An example of the piezoelectric substrate corresponds to the inner surface of the flow channel or is connected to the inner surface of the flow channel.

An example of the actuator is arranged downstream from the at least one zone of the flow channel. Thus, in an example, a cell exhibiting at least one modified movement is detectable at a point or in a region of the flow channel. This, in turn, facilitates diverting, by the actuator, the cell at a point or in a region of the flow channel further downstream. Consequently, data may be acquired during the travel of the cells through the flow channel. The data may then be used to accurately divert the correct cells further downstream. Collecting data relating to modified movements of the cells at several points of the flow channel or in a region of the flow channel facilitates increasing the accuracy of the sorting.

In an example, the detector may comprise:
a light source configured to illuminate a cell in the flow channel, such that an interference pattern is formed by interference between light being scattered by the illuminated cell and non-scattered light from the light source; and
an image sensor configured to detect an image series representing a time-sequence of interference patterns of the illuminated cell. Consequently, the detector may operate according to the principles of digital holographic imaging.

Such a detector facilitates high cell throughput. An example of a detector operating according to the principles of digital holographic imaging has a large field of view and, in a further example, does not require a lens between the image sensor and the flow channel. A further example of a detector operating according to the principles of digital holographic imaging has a large depth of focus. Consequently, an example of the detector can monitor a large area or a large volume, of the flow channel, for cells exhibiting modified movements. Monitoring a large area or volume simultaneously facilitates maintaining a high flow rate and a high cell throughput.

In an example, the non-scattered light from the light source is passed along a common optical path, with the light being scattered by the cells. Thus, in an example, the interference pattern is formed between scattered and non-scattered light at the image sensor in a so-called in-line holography set-up. Thus, the scattered and non-scattered light share the same light path. However, according to an alternative example, the non-scattered light is passed along a separate reference light path, which is combined with the light having been scattered by the cells before reaching the image sensor. In such a case, the light scattered by the cells is, for example, either forward or backward scattered light.

As an alternative to a detector operating according to the principles of digital holographic imaging, an example of the detector uses imaging with a lens to detect cells exhibiting modified movements. Thus, in an example, the detector detects an image series representing a time-sequence of a cell in the flow channel instead of a time-sequence of interference patterns of the cell.

An example of the detector further comprises a processor configured to analyze the time-sequence of interference patterns of the illuminated cell and detect the cell as exhibiting the at least one modified movement based on the time-sequence of interference patterns. An example of the processor generates the trigger signal.

An example of the image sensor is configured to detect a series of images representing a time-sequence of interference patterns of the illuminated cell. The image series represent both the illuminated cell in the at least one zone of the flow channel and the illuminated cell at the actuator. This facilitates ensuring, by the device, that the correct cell is that one that is diverted.

An example of the detector comprises multiple image sensors. The multiple image sensors facilitate imaging a larger area than a single image sensor could. In an example, each image sensor has its own light source. In another example, two or more image sensors share a light source. The field of view of an image sensor may or may not overlap with the field of view of another image sensor.

In an example where the detector comprises a light source configured to illuminate a cell in the flow channel, and comprises an image sensor configured to detect an image series representing a time-sequence of interference patterns of the illuminated cell, the light source is configured to emit at least partially coherent light. Coherent light facilitates improved interference visibility. An example of a coherent light source is a laser. However, it should be understood that partially coherent light can provide an interference pattern with sufficient visibility. An example of partially coherent light source corresponds to a light-emitting diode that emits light directly onto the flow channel or through a pinhole onto the flow channel. A coherent light source may provide better interference visibility but be more expensive, while a partially coherent light source may provide worse interference visibility but be less expensive.

An example of the device comprises a tracker configured to track the detected cell in the flow channel until it reaches the actuator. An example of the tracker is configured to monitor the position of a detected cell in the flow channel over a time-sequence from the detection of the cell exhibiting the modified movement until the cell reaches the actuator. An example of the tracker may be a processor monitoring the position of a detected cell based on information from an image sensor, e.g., an image sensor part of a digital holographic system.

The use of a tracker allows a modified movement of a cell to be detected at a distance from the actuator. This facilitates monitoring, by the detector, a large area or a large volume of the flow channel for cells exhibiting modified movements, which in turn facilitates achieving a high cell throughput. The modified movement need not be detected at the actuator but may be detected anywhere within the field of view of the detector. The cell may then be tracked such that it may be diverted once it reaches the actuator.

Further, the inclusion of a tracker in the device improves sorting accuracy. When the cell is tracked, several modified movements can be used to accurately identify a cell as belonging to a category. For example, when a cell exhibits two modified movements within a zone versus a single modified movement within the zone, the likelihood that the cell belongs to the cell category associated with the zone increases. Tracking facilitates counting the number of modified movements a cell exhibits. Tracking further facilitates monitoring the path of the cell.

In an example, the tracker and the detector are implemented as the same component, e.g., one component comprising a processor which performs both tracking and detecting tasks.

An example of the detector is configured to detect a cell exhibiting at least one modified movement based on:
i) at least two interference patterns in the time-sequence of interference patterns of the illuminated cell; and/or
ii) partial holographic reconstructions of at least two interference patterns in the time-sequence of interference patterns of the illuminated cell.

In some examples, two positions of the cell are sufficient to deduce that the cell exhibits a modified movement. For example, if a cell is positioned at a first point of a CMM surface and at a later time is positioned at a second point of the CMM surface at which the cell could not have moved by merely following the flow of liquid, it may be deduced that the cell has moved along the CMM surface, e.g., by rolling along the CMM surface in a manner indicating that the cell has an affinity to the CMM surface. This, in turn, would indicate that the cell belongs to the cell category associated with the zone having the CMM surface. In an example, the two positions or the movement between the two positions are derived from holographic reconstructions of at least two interference patterns in the time-sequence of interference patterns of the illuminated cell. In another example, the two positions or the movement between the two positions are derived from partial holographic reconstructions of at least two interference patterns in the time-sequence of interference patterns of the illuminated cell. In some instances, full reconstructions are not required to deduce that a modified movement has occurred. In another example, the two positions or the movement between the two positions are derived from at least two interference patterns in the time-sequence of interference patterns of the illuminated cell. Thus, no reconstruction may be needed at all. Skipping the step of holographic reconstruction. i.e., detecting the modified movement directly from the interference patterns, or performing partial holographic reconstructions, facilitates saving processing resources. When processing resources are saved, information from larger image sensors may be processed, which in turn facilitates higher cell throughput. The level of the holographic reconstructions may be selected according to an error acceptance level in the detection of the cells exhibiting modified movements. An example of a partial holographic reconstruction corresponds to a reconstruction with a lower resolution along the direction of the optical path through the flow channel, e.g., a reconstruction at a fixed focal distance within the flow channel. In contrast, an example of a full reconstruction corresponds to a reconstruction at several focal distances within the flow channel. In an example, focal distances within the flow channel cover the range from the top of the flow channel to the bottom of the flow channel.

Continuing with the above, an example of the tracker is configured to track the detected cell by illuminating the detected cell and tracking:
i) the interference pattern of the illuminated cell between successive images in the image series representing the time-sequence of interference patterns of the illuminated cell, and/or
ii) a partial holographic reconstruction of the interference pattern of the illuminated cell between successive images in the image series representing the time-sequence of interference patterns of the illuminated cell.

In an example, the position of the tracked cell for an image of the image series is derived from a holographic reconstruction of the interference pattern of the cell in the image. Alternatively, in another example, the position of the tracked cell for an image of the image series is derived from a partial holographic reconstruction of the interference pattern of the cell in the image. Alternatively, in another example, the position of the tracked cell for an image of the image series is derived from the interference pattern of the cell in the image. By avoiding the performing of holographic reconstruction, i.e., tracking the position of the detected cell directly from the interference patterns, or the performance of partial holographic reconstructions, processing resources may be saved. When processing resources are saved, information from larger image sensors may be processed, which in turn facilitates higher cell throughput.

In some example embodiments, the flow channel comprises more than one zone. An example of the flow channel comprises at least a first zone and a second zone, wherein a composition of the molecules coating at least one CMM surface of the first zone differs from a composition of the molecules coating at least one CMM surface of the second zone. In an example, within the flow channel, the second zone is arranged downstream from the first zone, and the actuator is arranged downstream from the second zone.

Providing several zones with different CMM surfaces facilitates sorting of the cells into multiple cell categories. Further, the cell sorting accuracy may be improved. The combination of information regarding the modified movements of cells in different zones facilitates complex analysis and sorting of the cells. In an example, the actuator services several zones, which facilitates performing complex analysis and sorting in a compact device.

Another example of the device is configured to detect a first movement and a second movement of a cell, where the first movement occurs in the first zone of the flow channel and a second movement occurs in the second zone of the flow channel. The first movement and second movement corresponds to movements modified by a CMM surface of the first zone or the second zone. The device is further configured to transmit a trigger signal based on the detection of the cell exhibiting the first movement and the second movement.

In an example, several zones with different CMM surfaces facilitate sorting of the cells into sub-categories. In one example, CMM surfaces of the first zone are coated by molecule A for detection of cells belonging to cell category A, and CMM surfaces of the second zone are coated by molecule B for detection of cells belonging to cell category B. Cells exhibiting modified movements in the first zone can then be categorized as belonging to cell category A. For example, cells exhibiting modified movements in the second zone are categorized as belonging to cell category B. Cells exhibiting modified movements in both the first zone and the second zone are categorized as belonging to cell sub-category AB, where cell sub-category AB corresponds to a sub-category of both cell category A and cell category B. In an example, the actuator diverts cells of cell category A to a first outlet and cells of cell category B to a second outlet. In another example, cells associated with category A that are not associated with sub-category AB are diverted to a first outlet. The cells associated with category B that are not associated with sub-category AB are diverted to a second outlet, and cells associated with sub-category AB are diverted to a third outlet. Cells that do not exhibit any modified movements in any zone may be diverted to a separate outlet.

In an example, at least one zone of the flow channel comprises at least one obstacle, thereby forming at least one zone of obstacles. The obstacle has a lateral surface configured to obstruct the path of the cell being carried by the flow of liquid. The lateral surface of the obstacle is comprised within the at least one CMM surface of the zone of obstacles.

In an example, the obstacle may be:
a pillar having an axis extending within the flow channel in a direction orthogonal to a main flow direction of the flow channel at a position of the pillar; and/or
a ridge having a height extending within the flow channel in a direction orthogonal to a main flow direction of the flow channel at a position of the ridge.

In an example, obstacles present in a zone increase the number of times a cell encounters a CMM surface during the travel through the zone. Consequently, the accuracy of the detection and, thereby, the sorting of cells can be improved. Further, the use of obstacles within the zones facilitates decreasing the size of the zones, which, in turn, facilitates reducing the size of the device. For example, if one zone comprising no obstacles is compared to a zone comprising obstacles, the zone comprising no obstacles may need to be longer in order for the same accuracy to be achieved.

According to a second aspect, a method for sorting biological cells comprises:
detecting a cell in a flow channel,
   wherein the flow channel is configured to pass a flow of liquid, the flow of liquid carrying the cells to be sorted, the flow channel comprising at least one zone, each zone of the flow channel being associated with a cell category,
   wherein each zone of the flow channel comprises at least one surface coated with molecules having an affinity to the cell category associated with the zone, the at least one surface of the zone being configured to modify, by the molecules coating the at least one surface, a movement of a cell belonging to the associated cell category of the zone as the cell carried by the flow of liquid passes the zone, whereby the at least one surface forms at least one cell movement modifying, CMM, surface, and
   wherein the detected cell is a cell exhibiting at least one modified movement in one zone of the flow channel; and
communicating a trigger signal, based on the detection of the cell exhibiting at least one modified movement, to an actuator configured to divert the detected cell within the flow of liquid in the flow channel.

Aspects associated with the device for sorting biological cells described above are equally applicable to the method for sorting biological according to the second aspect.

### BRIEF DESCRIPTION OF THE FIGURES

The above, as well as additional features, will be better understood through the following illustrative and non-limiting detailed description of example embodiments, with reference to the appended drawings. In the drawings, like reference numerals will be used for like elements unless stated otherwise.

FIG. 1 is a prior art device for label-free cell sorting.

FIG. 2 illustrates a device for sorting biological cells, in accordance with an example embodiment.

FIG. 3 illustrates a high-throughput device for sorting biological cells, in accordance with an example embodiment.

FIG. 4 illustrates a flow channel of a device for sorting biological cells, in accordance with an example embodiment.

FIG. 5 illustrates a device for sorting biological cells that comprises a detector, in accordance with an example embodiment.

FIG. 6 illustrates a device for sorting biological cells that comprises an actuator that creates an electric field within a flow of liquid to divert cells, in accordance with an example embodiment.

FIG. 7 illustrates a device for sorting biological cells that comprises an actuator that creates a surface acoustic wave within a flow of liquid to divert cells, in accordance with an example embodiment.

FIG. 8A illustrates obstacles in the form of ridges of a device for sorting biological cells, in accordance with an example embodiment.

FIG. 8B illustrates obstacles in the form of an undulating pattern that facilitate sorting biological cells, in accordance with an example embodiment.

FIG. 8C illustrates obstacles in the form of a slotted pattern that facilitate sorting biological cells, in accordance with an example embodiment.

FIG. 8D illustrates obstacles in the form of perforations that facilitate sorting biological cells, in accordance with an example embodiment.

FIG. 8E illustrates a profile of a slotted pattern that facilitates sorting biological cells, in accordance with an example embodiment.

FIG. 8F illustrates a profile of a grooved pattern that facilitates sorting biological cells, in accordance with an example embodiment.

FIG. 9 illustrates a flow chart for a method for sorting biological cells, in accordance with an example embodiment.

FIG. 10 illustrates a device that comprises zones having different fluid flow velocities that facilitate sorting biological cells belonging to different cell categories, in accordance with an example embodiment.

FIG. 11A illustrates the device of FIG. 10 with triangular shaped pillars, in accordance with an example embodiment.

FIG. 11B illustrates the device of FIG. 10 with teardrop shaped pillars, in accordance with an example embodiment.

FIG. 11C illustrates the device of FIG. 10 with star shaped pillars, in accordance with an example embodiment.

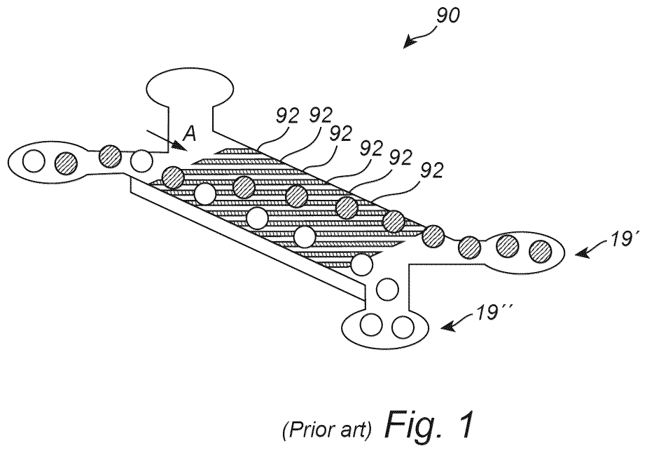

All the figures are schematic, not necessarily to scale, and generally only show parts that are necessary to elucidate example embodiments, wherein other parts may be omitted or merely suggested.

### DETAILED DESCRIPTION

Example embodiments will now be described more fully hereinafter with reference to the accompanying drawings. That which is encompassed by the claims may, however, be embodied in many different forms and should not be construed as limited to the embodiments set forth herein; rather, these embodiments are provided by way of example. Furthermore, like numbers refer to the same or similar elements or components throughout.

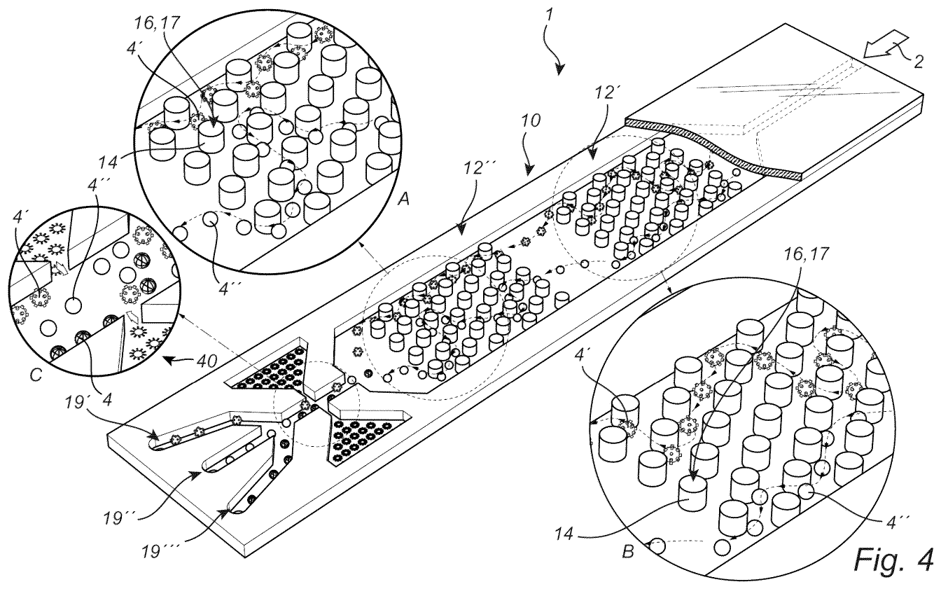

FIG. 4 illustrates a flow channel 10 of a device 1 for sorting biological cells 4. The flow channel 10 is configured to pass a flow 2 of liquid carrying the cells 4 to be sorted. An example of the flow channel 10 is a microfluidic channel. An example of the flow channel 10 is defined by conventional lithography and sealed with a transparent cover slide, e.g., glass, PDMS, or polycarbonate. In an example, the top part of the flow channel 10 is covered with a cover slide, e.g., a glass slide, bonded to the PDMS.

The illustrated flow channel 10 comprises two zones 12. The flow 2 of liquid and the cells 4 first passes a first zone 12′ and then a second zone 12″. Each zone 12 comprises obstacles 16, and in the figure, obstacles 16 in the form of pillars 17.

In the illustrated device 1, the sidewalls of the pillars 17 form lateral surfaces that obstruct the path of the cells 4. Thus, the cells 4 repeatedly hit the sidewalls of the pillars 17 during their travel through the flow channel 10. Further, the sidewalls of the pillars 17 form cell movement modifying (CMM) surfaces 14. The CMM surfaces 14 comprise molecules having an affinity to a cell category. In the illustrated flow channel 10, the CMM surfaces 14 of the first zone 12′ are coated by molecules having an affinity to cells 4′ of a first category. Thus, the first zone 12′ may be considered to be associated with the first category. In the illustrated flow channel 10, the CMM surfaces 14 of the second zone 12″ are coated by molecules having an affinity to cells 4″ of a second category. Thus, the second zone 12″ may be considered to be associated with the second category.

Examples of the molecules of the CMM surfaces 14 correspond to antibodies, aptamers, lectins, etc. For instance, in an example, the molecules of the CMM surface 14 of the first zone 12′ are anti-CD4 antibodies and the molecules of the CMM surface 14 of the second zone 12″ are anti-CD8 antibodies. In this case, a first cell category represents cells 4′ having a cell membrane comprising CD4 antigens and a second cell category represents cells 4″ having a cell membrane comprising CD8 antigens.

As illustrated in FIG. 4, examples of the molecules of the CMM surfaces 14 modify the movement of the cells 4. The modified movements are associated with the type of path the cells 4 take as they interact with the CMM surfaces 14 of a zone 12. In FIG. 4 a cell 4′ of the first category takes a more undulating path in the first zone 12′ than in the second zone 12″. Analogously, a cell 4″ of the second category takes a more undulating path in the second zone 12″ than in the first zone 12′.

In an example, the molecules bind and release a cell 4 belonging to the associated cell category of the zone 12. For instance, in an example, an anti-CD4 antibody binds to a cell 4 having a cell membrane comprising CD4 antigens and subsequently releases the cell. Thus, the movements of a cell 4′ of the first category moving through the first zone 12′ may change in a characteristic way that makes it possible to identify the cell as belonging to the first category. The movements of a cell 4″ of the second category moving through the second zone 12″ may similarly change. When a cell belongs to a category associated with a zone, the cell may, in that zone, exhibit, e.g., a bind and release event, a halt of motion, a change of speed, a change of direction, or a change of path. For example, a cell that is observed to stick for a short time to a pillar in the first zone 12′ may be regarded as a cell 4′ of the first category. Alternatively, a cell that is observed to stick for a short time to a threshold number, e.g., 10, of pillars in the first zone 12′ may be regarded as a cell 4′ of the first category. In another example, cells that exhibit bind-roll-release type of movements in the first zone 12′ may be regarded as a cell 4′ of the first category. FIG. 4 illustrates a cell 4′ of the first category, which, in the first zone 12′, follows the curvature of the pillars 17 to an extent that indicates that the cell does not merely follow the flow 2 of liquid.

In an example, a modified movement is detected at a specific location of the zone 12, e.g., by the cell 4 following the CMM surface 14 of a single pillar 17 of the zone 12 in a characteristic way. In another example, a modified movement is detected over the entire zone 12 or over an area of the zone 12, e.g., by the cell 4 taking an undulating path when interacting with several obstacles 16 of the zone 12.

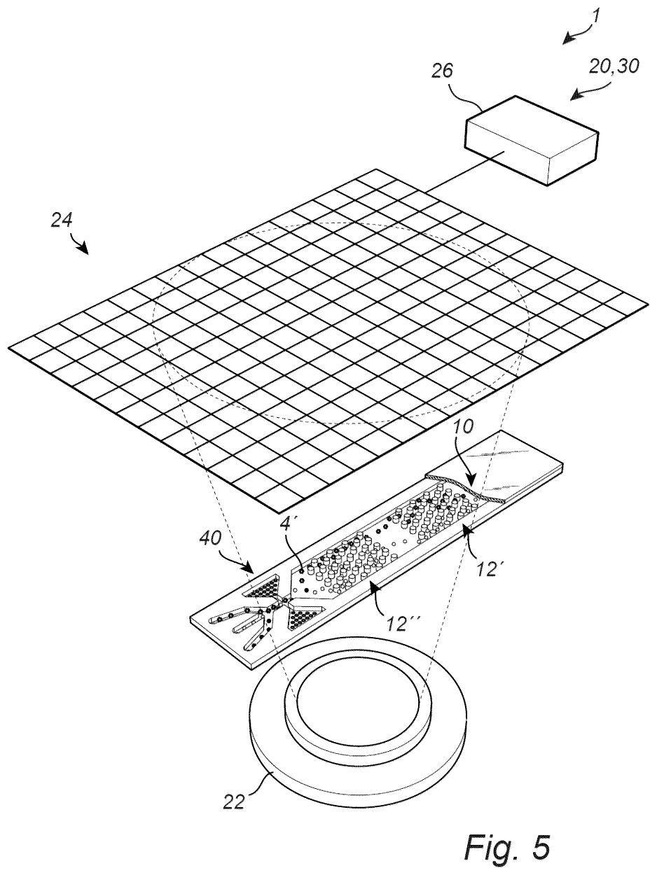

FIG. 5 illustrates a device 1 with a detector 20 comprising a light source 22 and an image sensor 24. The illustrated light source 22 illuminates the zones 12 of the flow channel 10. The flow channel 10 of the device 1 in FIG. 5 is the flow channel 10 depicted in FIG. 4.

The illustrated light source 22 is configured to illuminate the cells 4 as they pass through the flow channel 10. An example of the light source 22 is a coherent light source 22, e.g., a laser, or a partially coherent light source 22, e.g., a light-emitting diode or a light-emitting diode with a pinhole or aperture. As the cells 4 are illuminated, an interference pattern is formed on the image sensor 24. An example of the image sensor 24 comprises a plurality of photo-sensitive elements configured to detect incident light, such as a CCD or CMOS camera. In an example, the image sensor 24 acquires a time-sequence of image frames of the changing interference pattern as cells 4 pass the image sensor 24. In the figure, the light source 22 and the image sensor 24 are arranged on opposite sides of the flow channel 10. The flow channel 10 is herein at least partially transparent such that the light may travel through the flow channel 10.

The illustrated detector 20 further comprises a processor 26 configured to detect a cell exhibiting at least one modified movement based on the time-sequence of interference patterns. In an example, the processor 26, performs a holographic reconstruction of two interference patterns in the time-sequence of interference patterns. The two reconstructions may depict, e.g., a cell in contact with a CMM surface 14. In an example, if both reconstructed images show the cell 4 at the same location, then the cell is determined to be stuck to the CMM surface and therefore has an affinity to the CMM surface 14. If both reconstructed images show the cell 4 in contact with the CMM surface, but at different locations, the cell is determined to be stuck to the CMM surface 14 and thereafter rolled along the CMM surface 14. In either case, the cell 4 may be detected as exhibiting a modified movement.

The processor 26 may not necessarily perform a full holographic reconstruction of two interference patterns in the time-sequence of interference patterns. In some examples, the cell 4 is detected as exhibiting a modified movement based on two or more interference patterns in the time-sequence of interference patterns or partial holographic reconstructions of the two or more interference patterns.

An example of the device 1 comprises a tracker 30 configured to track the detected cell 4 in the flow channel 10 until it reaches the actuator 40. In FIG. 5, the tracker 30 is implemented using the same light source 22, image sensor 24, and processor 26 as the detector 20. An example of the processor 26 is configured to track a detected cell 4, e.g., follow the position of the cell 4 through the flow channel 10 until the cell 4 reaches the actuator 40, based on the time-sequence of interference patterns of the illuminated cell 4. An example of the processor 26 tracks the cell 4 based on holographic reconstructions of the time-sequence of interference patterns, based on partial holographic reconstructions of the time-sequence of interference patterns, or based on the interference patterns without any reconstruction.

As illustrated in FIG. 5, in an example, the light source 22 and image sensor 24 are arranged to cover the actuator 40 such that cells 4 may be tracked all the way to the actuator 40.

In an example, the processor 26 transmits a trigger signal to the actuator 40, e.g., via an electrical wire (not shown), such that the actuator 40 may divert the detected cell 4 within the flow 2 of liquid in the flow channel 10. An example of the actuator 40 is activated as the cell 4 passes the actuator 40 based on the tracking of the cell 4.

An example of the processor 26 detects a first and a second movement of a cell 4, the first movement being in the first zone 12′ of the flow channel 10 and the second movement being in the second zone 12″ of the flow channel 10. For example, in FIG. 5, a cell 4′ of a first category exhibits an undulating path in the first zone 12′ and a fairly straight path in the second zone 12″. The processor 26, in this example, categorizes the cell as belonging to the first category but not the second category. The trigger signal is then be based on the detection of the cell exhibiting the first and second movement. The cell 4 may be tracked between the detection of the first movement and the detection of the second movement.

The actuator 40 may be configured in various ways. In FIG. 5 the actuator is a jet flow actuator. An example of a jet flow actuator comprises at least one array of heaters. In FIG. 5 there are two arrays of heaters, one on each side of the flow channel 10. When actuated heaters generate a steam bubble at their surfaces, which displace the liquid and thereby apply a gentle push to the passing cell. In FIG. 5 each array of heaters is arranged in a reservoir adjacent to the flow channel 10 such that the combined displaced volume of the heaters passes the inlet between the reservoir and the flow channel 10. Jet flow actuators are described in patent application WO18122215, which is hereby included by reference.

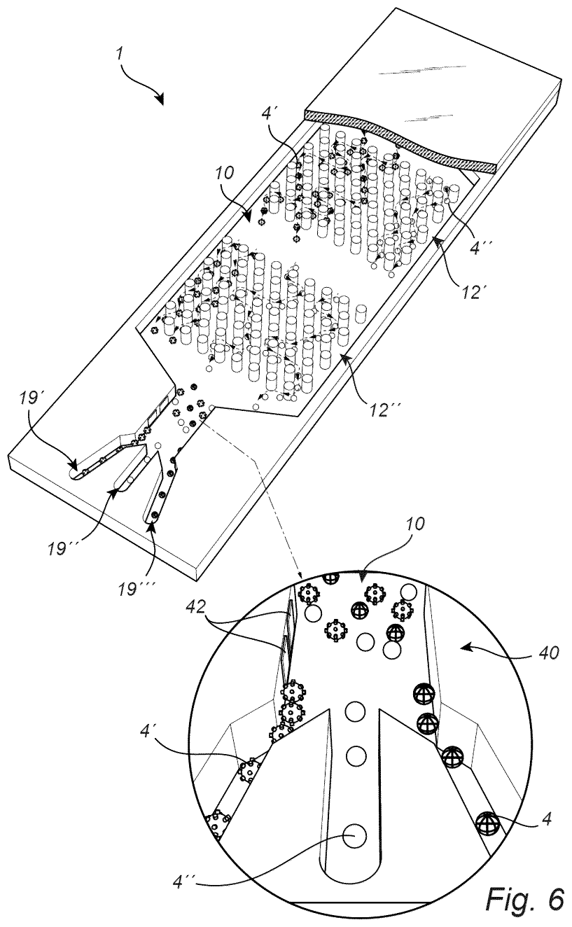

FIG. 6 illustrates a device 1 wherein the actuator 40 is configured to divert the detected cell 4 within the flow of liquid in the flow channel 10 by creating an electric field within the flow 2 of liquid. The illustrated device 1 comprises two electrodes 42 on the same side of the flow channel 10. In an example, a non-uniform electric field is created within the flow 2 of liquid. In an example, an electric field, e.g., an alternating electric field between the two electrodes 42, divert cells away from or towards the strongest field intensity through dielectrophoresis. The strength and/or direction of the force exerted on the cells may be dependent on cell type and/or the frequency of the electric field. The mechanism of dielectrophoresis and actuators operating according to this mechanism is described by C. Wyatt Shields IV et al. in Lab on a Chip 15, no. 5 (2015): 1230-1249, which is hereby incorporated by reference. In FIG. 6 the electrodes 42 are in contact with the liquid in the flow channel 10. However, the electrodes 42 do not necessarily need to be in contact with the liquid in the flow channel 10.

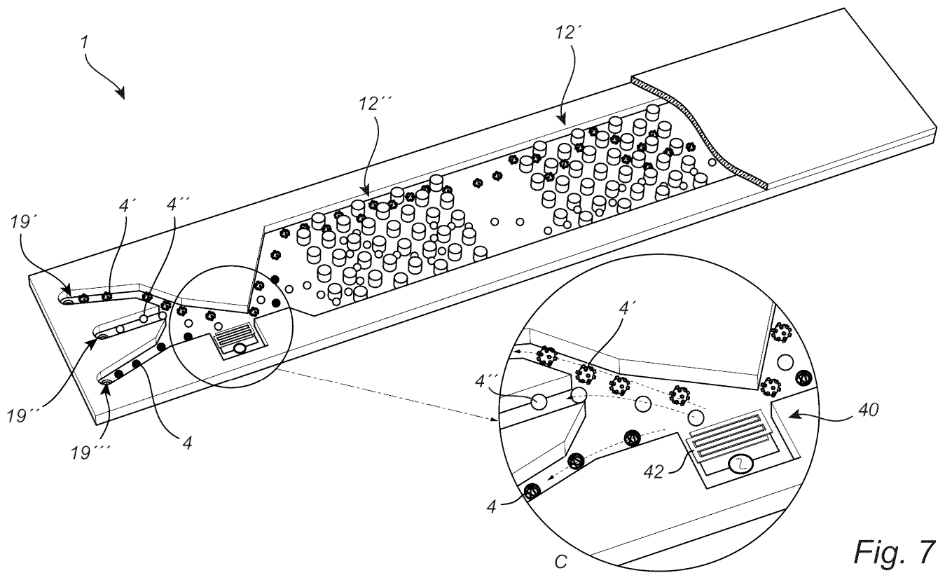

FIG. 7 illustrates a device 1 wherein the actuator 40 is configured to divert the detected cell 4 within the flow of liquid in the flow channel 10 by creating a surface acoustic wave along an inner surface of the flow channel. The illustrated device 1 comprises two electrodes 42, the electrodes being interdigitated and arranged on a surface extending across the flow channel 10. In FIG. 7, the interdigitated electrodes 42 are arranged on a surface that forms the bottom of the flow channel 10. An example of the surface on which the interdigitated electrodes 42 are arranged is made of a piezoelectric material, e.g., lithium niobate. Thus, in an example, an alternating voltage across the interdigitated electrodes 42 is converted into a surface acoustic wave in the piezoelectric material. The surface acoustic wave is, in turn, transferred into a wave in the liquid in the flow channel 10, which then diverts a cell 4. In FIG. 7, the electrodes 42 are in contact with the liquid in the flow channel 10. However, the electrodes 42 do not necessarily need to be in contact with the liquid in the flow channel 10 as described, e.g., by C. Wyatt Shields IV et al. in Lab on a Chip 15, no. 5 (2015): 1230-1249.

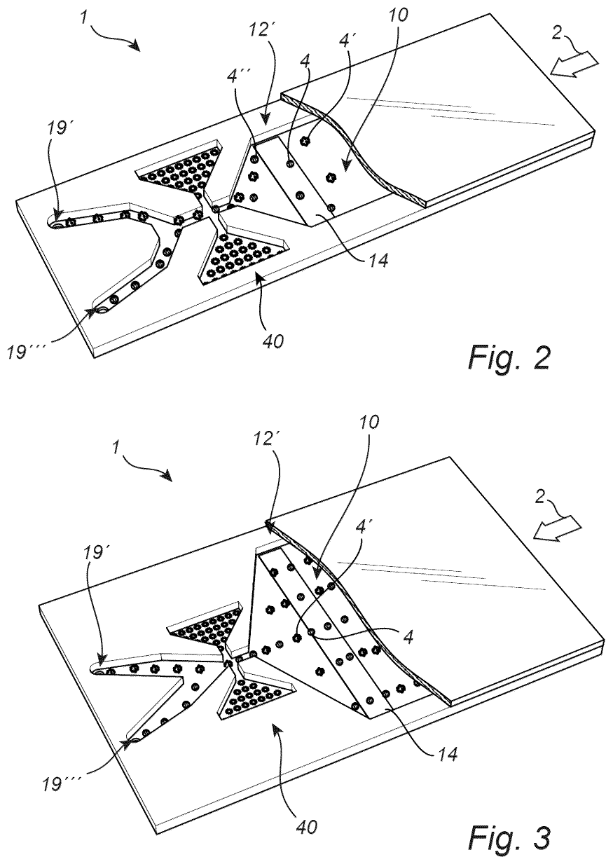

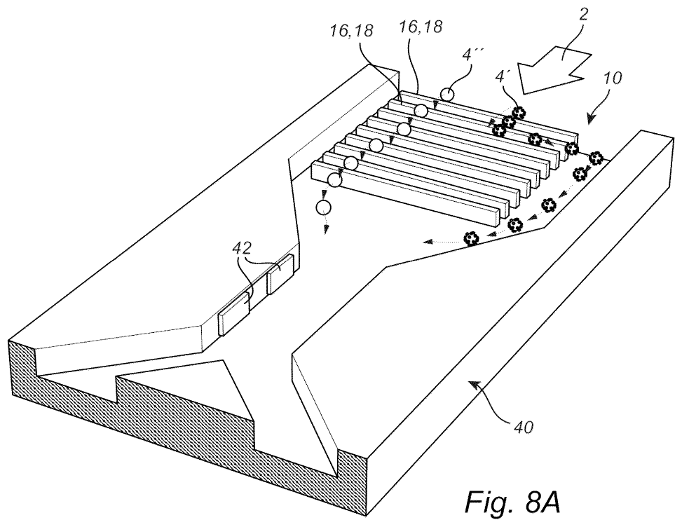

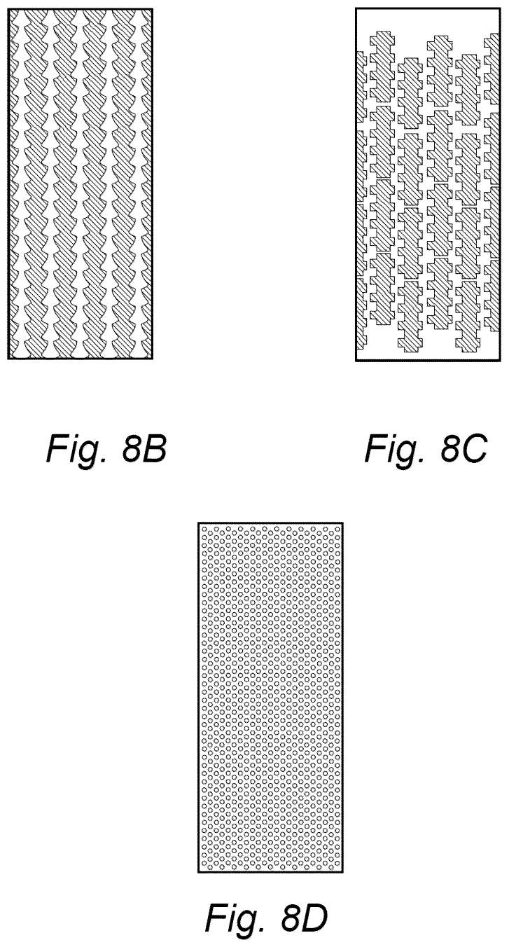

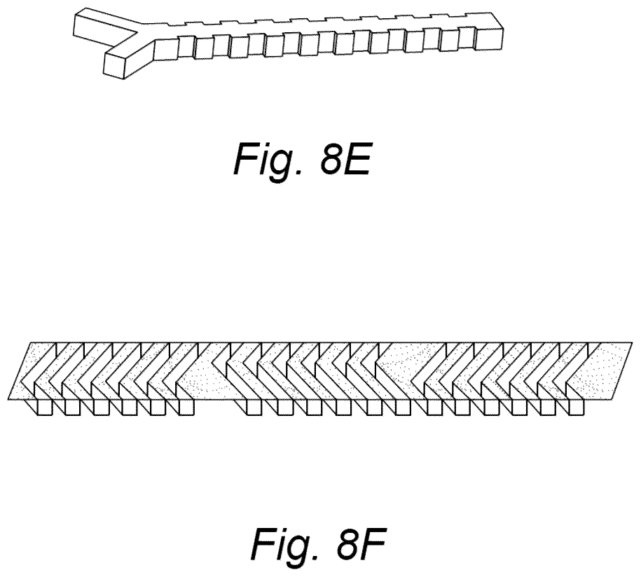

As previously discussed, an example of the CMM surface 14 corresponds to a surface of a sidewall of the flow channel 10 coated with CMM molecules, as illustrated in FIGS. 2 and 3. Another example of a CMM surface 14 is a lateral surface of an obstacle 16, such as a pillar 17. Other obstacles 16 may, of course, alternatively be used. FIG. 8A illustrates obstacles 16 in the form of ridges 18. The illustrated ridges 18 extend from the bottom of the flow channel 10 to form a steeplechase for the cells 4. The ridges 18 are coated with molecules having an affinity to a first cell category. Thus, the surfaces of the ridges 18 form CMM surfaces 14. Cells 4′ of the first category preferentially stick to the ridges 18 and exhibit a modified movement where they move to either side of the flow channel 10. In contrast, cells 4″ of a second cell category are largely unaffected by the ridges 18. Thus, cells 4′ of the first category may be detected based on their movements. When a detected cell 4′ reaches the actuator 40 it may be diverted such that it is separated from other cells 4. FIGS. 8B-8F illustrate other examples of obstacles that may be formed in one or more surfaces of the flow channel 10.

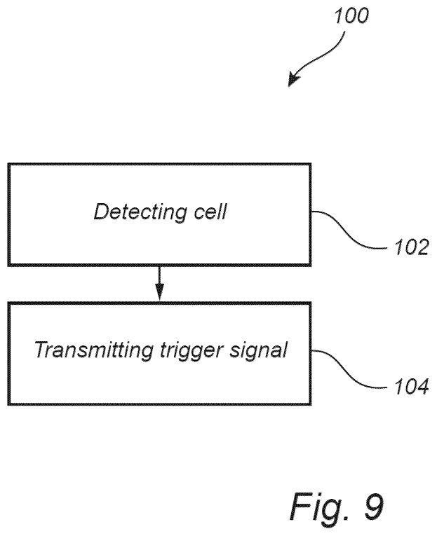

FIG. 9 illustrates a flow chart of a method 100 for sorting biological cells. According to the method 100, a cell 4 in a flow channel 10 is detected 102, the detected cell 4 being a cell 4 exhibiting at least one modified movement in one zone 12 of the flow channel 10. A trigger signal, based on the detection 102 of the cell 4, is subsequently transmitted 104 to an actuator 40. In an example, the actuator 40 then diverts the detected cell 4 within the flow 2 of liquid in the flow channel 10. An example of the method 100 is performed using one of the devices 1 described above. For instance, an example of the method 100 is performed by a processor 26 in one of the devices 1 described above.

As noted above in FIG. 4, some examples of a flow channel 10 of a device 1 sorting biological cells comprise multiple zones (12′, 12″). Some examples of the zones (12′, 12″) are configured to facilitate sorting biological cells 4 belonging to different cell categories that are in a fluid that flows through the flow channel 10. Some examples of the zones (12′, 12″) comprise pillars 17 that comprise CMM surfaces 14 having molecules that have an affinity to the different categories of cells. For example, a first group of pillars comprises a CMM surface 14 that comprises antibodies that have an affinity to cells having a particular combination of receptors/antigens, a second group of pillars comprises a CMM surface 14 that comprises antibodies that have an affinity to cells having a different combination of receptors/antigens, etc.

The adsorbing force between a particular CMM surface 14 and a cell having corresponding receptors is generally proportional the density of receptors on the surface of the cell. This force influences the attachment and detachment rates (denoted K_{ON} and K_{OFF} rate, respectively) of the cell for a particular fluid flow velocity. The respective K_{ON} and K_{OFF} rate for the cell affects the interaction profile of the cell (e.g., the direction of movement of the cell through the pillars 17). The interaction profile facilitates determining the category to which the cell belongs.

When the adsorbing force associated with a cell belonging to a particular category is too low, the corresponding interaction profile of the cell may lack the distinctiveness required to facilitate determining the category to which the cell belongs. On the other hand, when the adsorbing force is too high, the cell may bind to the CMM surface 14 for an excessive amount of time, which can result in cell blockages within the zones (12′, 12″). The blockages can impede the flow of cells through the zones (12′, 12″). This issue can be mitigated by balancing the fluid flow velocity with the adsorbing force associated with cells of different categories.

For instance, some examples of the device utilize a single fluid flow velocity, and the densities of the antibodies on the CMM surfaces 14 of the pillars 17 are selected to balance the fluid flow velocity with respective adsorbing forces associated with cells in the fluid. For example, the density of antibodies on the CMM surface 14 of a first group of pillars 17 (e.g., pillars 17 in a first zone 12′) that have an affinity to cells belonging to a first category is different than the density of antibodies on the CMM surface 14 of a second group of pillars 17 (e.g., pillars 17 in a second zone 12″) that have an affinity to cells belonging to a second category.

In this regard, cells belonging to the first category may have a higher receptor density than cells belonging to the second category. If the antibody densities on the respective CMM surfaces 14 are the same, the adsorbing force between the cells belonging to the first category and the corresponding CMM surfaces 14 (e.g., in the first zone 12′) may be higher than the adsorbing force between the cells belonging to the second category and the corresponding CMM surfaces 14 (e.g., in the second zone 12″). This could result in the formation of cell blockages within, for example, the first zone 12′ for a particular velocity of fluid flow through the device 1. Lowering the antibody density of the CMM surfaces 14 of the first group of pillars 17 decreases the adsorbing force between these surfaces and the first category of cells, thereby mitigating this issue.

In some examples, particular antibodies are selected for application on the CMM surfaces 14 to mitigate the issues above. In this regard, cells belonging to particular cell categories may comprise different antigens, and the density of these antigens on these cells may be different. For example, the density associated with a first antigen of a particular cell may be relatively high (e.g., few tens of thousands or more antigens per cell), and the density associated with a second antigen of the cell be relatively low (e.g., few thousands or less antigens per cell). The adsorbing force between the first antigens of the cell and a CMM surface 14 comprising corresponding antibodies may be higher than the adsorbing force between the second antigens of the cell and a CMM surface 14 comprising corresponding antibodies. Therefore, the adsorbing force can be increased or decreased as required by selecting an appropriate antibody to thereby balance the corresponding absorbing force with the fluid flow velocity to mitigate the issues noted above.

Further, some examples of the device comprise CMM surfaces 14 that comprise combinations of antibodies that correspond with different cell antigens having different antigen densities. In these examples, the adsorbing force can be increased or decreased as required by selecting an appropriate combination of antibodies that balances the corresponding absorbing force with the fluid flow velocity to mitigate the issues noted above.

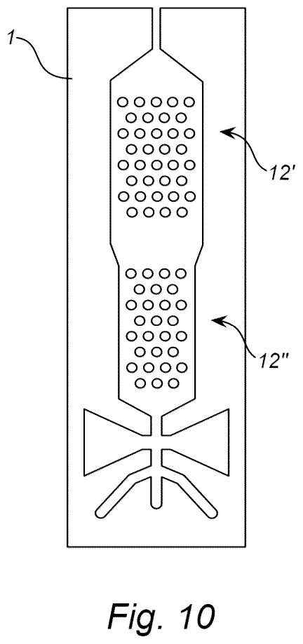

Some examples of the device 1 are configured to mitigate the issues noted above by providing zones having different fluid flow velocities. For example, a particular fluid flow may comprise cells belonging to a first category and cells belonging to a second category. The cells of the second category may have a higher receptor/antigen density than the cells belonging to the first category. The antibody densities on CMM surfaces 14 of pillars in the first zone 12′ and the second zone 12″ of the device for detecting the interaction profiles of cells belonging to the first category and the second category, respectively, may be the same. The adsorbing force, in this case, between the CMM surfaces 14 of the pillars in the second zone 12″ and the cells belonging to the second category may be higher than the adsorbing force between the CMM surfaces 14 of the pillars in the first zone 12′ and the cell belonging to the first category. This difference could result in cell blockages in the second zone 12″ for a particular/single fluid flow velocity. This issue can be mitigated, however, by increasing the fluid flow velocity in the second zone 12″. For example, as illustrated in FIG. 10, some examples of the device 1 comprise a second zone 12″ having a width that is smaller than the width of the first zone 12′. The decreased width of the second zone 12″ causes a corresponding increase in the fluid flow velocity in the second zone 12″. The increased fluid velocity compensates for the increased adsorbing force between cells belonging to the second category and the CMM surfaces 14 of the pillars 17 in the second zone 12″.

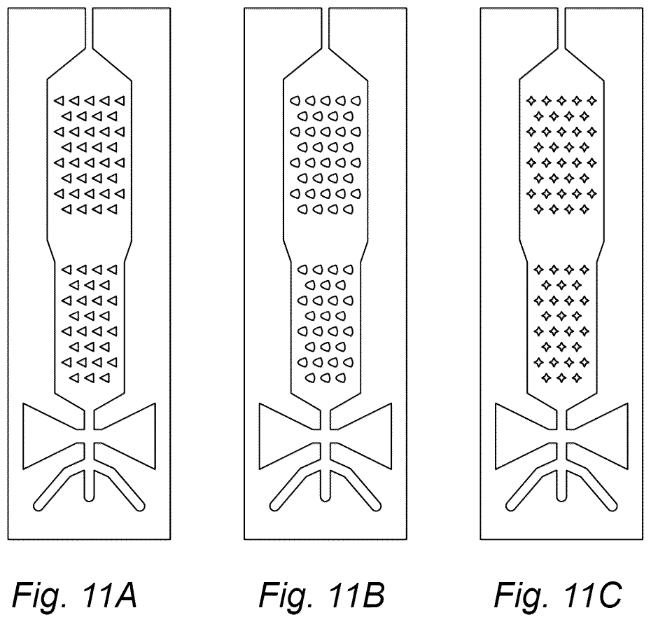

As noted above, some examples of the device 1 comprise pillars and the pillars comprise CMM surfaces 14. Some examples of the pillars are illustrated in the figures as having generally cylindrical shapes. However, other shapes are contemplated. For example, as illustrated in FIGS. 11A-11C, examples of the pillars may have a triangular shape, teardrop shape, star shape, etc. For a particular diameter, different shapes may inherently comprise different surface areas, which may in some instances facilitate providing pillars with different cellular affinities. For example, a first zone may comprise a particular number of pillars having cylindrical shapes and a second zone may comprise the same number pillars but having a triangular shape. The triangular shaped pillars may comprise more surface area and, therefore, a larger CMM surface area for binding to cells.

Additional examples of devices that facilitate the categorization of biological cells are elucidated in the following examples.
Example 1. A device for sorting biological cells comprising:
a flow channel configured to pass a flow of liquid, wherein:
   the flow of liquid carries the biological cells to be sorted,
   the flow channel comprises at least two zones,
   each zone of the flow channel is associated with a cell category, and
   each zone of the flow channel comprises at least one surface coated with molecules having an affinity and specificity to the cell category associated with the zone, the at least one surface of the zone being configured to modify, by the molecules coating the at least one surface, a movement of a biological cell belonging to an associated biological cell category of the zone as the cell, carried by the flow of liquid, passes the zone, whereby the at least one surface forms at least one cell movement modifying (CMM) surface, wherein a density of molecules on the at least one surface of a first zone is configured to be different from a density of molecules on the at least one surface of a second zone to facilitate categorizing biological cells having different receptor densities via a common volumetric flow rate of biological cells through the first zone and the second zone;
a detector configured to detect a biological cell exhibiting at least one modified movement in one zone of the flow channel, and to communicate a trigger signal based on the detection of the biological cell exhibiting the at least one modified movement; and
an actuator configured to divert, based on the trigger signal, the detected biological cell within the flow of liquid in the flow channel.
Example 2. A device for sorting biological cells comprising:
a flow channel configured to pass a flow of liquid, wherein:
   the flow of liquid carries the biological cells to be sorted,
   the flow channel comprises at least two zones,
   each zone of the flow channel is associated with a cell category, and
   each zone of the flow channel comprises at least one surface coated with molecules having an affinity and specificity to the cell category associated with the zone, the at least one surface of the zone being configured to modify, by the molecules coating the at least one surface, a movement of a biological cell belonging to an associated biological cell category of the zone as the cell, carried by the flow of liquid, passes the zone, whereby the at least one surface forms at least one cell movement modifying (CMM) surface, wherein molecules on the at least one surface of a first zone and molecules on the at least one surface of a second zone are selected so that an affinity of a first category of biological cells to the at least one surface of a first zone is substantially similar to an affinity of a second category of biological cells to the at least one surface of a second zone to facilitate categorizing biological cells having different receptor densities via a common volumetric flow rate of biological cells through the first zone and the second zone;
a detector configured to detect a biological cell exhibiting at least one modified movement in one zone of the flow channel, and to communicate a trigger signal based on the detection of the biological cell exhibiting the at least one modified movement; and
an actuator configured to divert, based on the trigger signal, the detected biological cell within the flow of liquid in the flow channel.
Example 3. A device for sorting biological cells comprising:
a flow channel configured to pass a flow of liquid, wherein:
   the flow of liquid carries the biological cells to be sorted,
   the flow channel comprises at least two zones,
   each zone of the flow channel is associated with a cell category, and
   each zone of the flow channel comprises at least one surface coated with molecules having an affinity and specificity to the cell category associated with the zone, the at least one surface of the zone being configured to modify, by the molecules coating the at least one surface, a movement of a biological cell belonging to an associated biological cell category of the zone as the cell, carried by the flow of liquid, passes the zone, whereby the at least one surface forms at least one cell movement modifying (CMM) surface, wherein dimensions of the first zone and the second zone are configured to cause the velocity of the liquid that carries the biological cells to be different in the first zone and the second zone to facilitate categorizing biological cells having different affinities to the at least one surface of the first zone and the at least one surface of the second zone;
a detector configured to detect a biological cell exhibiting at least one modified movement in one zone of the flow channel, and to communicate a trigger signal based on the detection of the biological cell exhibiting the at least one modified movement; and
an actuator configured to divert, based on the trigger signal, the detected biological cell within the flow of liquid in the flow channel.
Example 4. The device of examples 1-3, wherein the at least one zone of the flow channel comprises at least a first zone and a second zone,
wherein a composition of the molecules coating the at least one CMM surface of the first zone differs from a composition of the molecules coating the at least one CMM surface of the second zone.
Example 5. The device of example 4, wherein the device is further configured to:
detect a first movement and a second movement of a biological cell, wherein the first movement occurs in the first zone of the flow channel and a second movement occurs in the second zone of the flow channel, wherein at least one of the first movement and the second movement is a movement modified by a CMM surface of the first or second zone; and
communicate the trigger signal based on the detection of the biological cell exhibiting the first movement and the second movement.
Example 6. The device of example 5, wherein:
at least one zone of the flow channel comprises at least one obstacle, thereby forming at least one zone of obstacles, the at least one obstacle having a lateral surface configured to obstruct a path of the biological cell carried by the flow of liquid, the lateral surface of the at least one obstacle of the at least one zone of obstacles being comprised within the least one CMM surface of the at least one zone of obstacles.
Example 7. The device of example 6, wherein the at least one obstacle of the at least one zone of obstacles of the flow channel corresponds to:
a pillar having an axis that extends within the flow channel in a direction orthogonal to a main flow direction of the flow channel at a position of the pillar.
Example 8. The device of example 6, wherein the at least one obstacle of the at least one zone of obstacles of the flow channel corresponds to:
a ridge having a height extending within the flow channel in a direction orthogonal to a main flow direction of the flow channel at a position of the ridge.
Example 9. The device of example 6, wherein the molecules coating at least one CMM surface of the at least one zone of the flow channel comprise at least one of: antibodies or aptamers.
Example 10. The device of example 6, wherein the at least one CMM surface of at least one zone of the flow channel is configured to modify the movement of the biological cell belonging to the associated cell category of the zone by binding and releasing the biological cell belonging to the associated cell category of the zone.
Example 11. The device of examples 1-3, the device further comprising:
a tracker configured to track the detected biological cell in the flow channel until the biological cell reaches the actuator.
Example 12. The device of example 11, wherein the detector further comprises:
a light source configured to illuminate a biological cell in the flow channel such that an interference pattern is formed by interference between light being scattered by the illuminated biological cell and non-scattered light from the light source; and
an image sensor configured to detect an image series that represents a time-sequence of interference patterns of the illuminated biological cell.
Example 13. The device of example 12, wherein the detector is configured to detect a biological cell exhibiting at least one modified movement based on at least two interference patterns in the time-sequence of interference patterns of the illuminated biological cell.
Example 14. The device of example 12, wherein the detector is configured to detect a biological cell exhibiting at least one modified movement based on partial holographic reconstructions of at least two interference patterns in the time-sequence of interference patterns of the illuminated biological cell.
Example 15. The device of example 12, wherein the tracker is configured to track the detected biological cell by illuminating the detected biological cell and tracking the interference pattern of the illuminated biological cell between successive images in the image series representing the time-sequence of interference patterns of the illuminated biological cell.
Example 16. The device of claim 12, wherein the tracker is configured to track the detected biological cell by illuminating the detected biological cell and tracking a partial holographic reconstruction of the interference pattern of the illuminated biological cell between successive images in the image series representing the time-sequence of interference patterns of the illuminated biological cell.
Example 17. The device of example 12, wherein the light source is configured to emit at least partially coherent light.
Example 18. The device of examples 1-3, wherein the actuator is configured to divert the detected biological cell within the flow of liquid in the flow channel by creating:
an electric field within the flow of liquid;
a jet flow within the flow of liquid by heating the liquid; or
a surface acoustic wave along an inner surface of the flow channel.
Example 19. The device of examples 1-3, wherein:
at least one zone of the flow channel comprises at least one obstacle, thereby forming at least one zone of obstacles, the at least one obstacle having a lateral surface configured to obstruct a path of the biological cell carried by the flow of liquid, the lateral surface of the at least one obstacle of the at least one zone of obstacles being comprised within the least one CMM surface of the at least one zone of obstacles.
Example 20. The device of examples 1-3, wherein the molecules coating at least one CMM surface of the at least one zone of the flow channel comprise at least one of: antibodies or aptamers.
Example 21. The device of examples 1-3, wherein the at least one CMM surface of at least one zone of the flow channel is configured to modify the movement of the biological cell belonging to the associated cell category of the zone by binding and releasing the biological cell belonging to the associated cell category of the zone.

While some embodiments have been illustrated and described in detail in the appended drawings and the foregoing description, such illustration and description are to be considered illustrative and not restrictive. Other variations to the disclosed embodiments can be understood and effected in practicing the claims, from a study of the drawings, the disclosure, and the appended claims. The mere fact that certain measures or features are recited in mutually different dependent claims does not indicate that a combination of these measures or features cannot be used. Any reference signs in the claims should not be construed as limiting the scope.

1. A device for sorting biological cells comprising:
a flow channel configured to pass a flow of liquid, wherein:
the flow of liquid carries the biological cells to be sorted,
the flow channel comprises at least one zone,
each zone of the flow channel is associated with a cell category, and
each zone of the flow channel comprises at least one surface coated with molecules having an affinity and specificity to the cell category associated with the zone, the at least one surface of the zone being configured to modify, by the molecules coating the at least one surface, a movement of a biological cell belonging to an associated biological cell category of the zone as the cell, carried by the flow of liquid, passes the zone, whereby the at least one surface forms at least one cell movement modifying (CMM) surface;
a detector configured to detect a biological cell exhibiting at least one modified movement in one zone of the flow channel, and to communicate a trigger signal based on the detection of the biological cell exhibiting the at least one modified movement; and
an actuator configured to divert, based on the trigger signal, the detected biological cell within the flow of liquid in the flow channel.

2. The device of claim 1, wherein the at least one zone of the flow channel comprises at least a first zone and a second zone,
wherein a composition of the molecules coating the at least one CMM surface of the first zone differs from a composition of the molecules coating the at least one CMM surface of the second zone.

3. The device of claim 2, wherein the device is further configured to:
detect a first movement and a second movement of a biological cell, wherein the first movement occurs in the first zone of the flow channel and a second movement occurs in the second zone of the flow channel, wherein at least one of the first movement and the second movement is a movement modified by a CMM surface of the first or second zone; and
communicate the trigger signal based on the detection of the biological cell exhibiting the first movement and the second movement.

4. The device of claim 3, wherein:
at least one zone of the flow channel comprises at least one obstacle, thereby forming at least one zone of obstacles, the at least one obstacle having a lateral surface configured to obstruct a path of the biological cell carried by the flow of liquid, the lateral surface of the at least one obstacle of the at least one zone of obstacles being comprised within the least one CMM surface of the at least one zone of obstacles.

5. The device of claim 4, wherein the at least one obstacle of the at least one zone of obstacles of the flow channel corresponds to:
a pillar having an axis that extends within the flow channel in a direction orthogonal to a main flow direction of the flow channel at a position of the pillar.

6. The device of claim 4, wherein the at least one obstacle of the at least one zone of obstacles of the flow channel corresponds to:
a ridge having a height extending within the flow channel in a direction orthogonal to a main flow direction of the flow channel at a position of the ridge.

7. The device of claim 4, wherein the molecules coating at least one CMM surface of the at least one zone of the flow channel comprise at least one of: antibodies or aptamers.

8. The device of claim 4, wherein the at least one CMM surface of at least one zone of the flow channel is configured to modify the movement of the biological cell belonging to the associated cell category of the zone by binding and releasing the biological cell belonging to the associated cell category of the zone.

9. The device of claim 1, the device further comprising:
a tracker configured to track the detected biological cell in the flow channel until the biological cell reaches the actuator.

10. The device of claim 9, wherein the detector further comprises:
a light source configured to illuminate a biological cell in the flow channel such that an interference pattern is formed by interference between light being scattered by the illuminated biological cell and non-scattered light from the light source; and
an image sensor configured to detect an image series that represents a time-sequence of interference patterns of the illuminated biological cell.

11. The device of claim 10, wherein the detector is configured to detect a biological cell exhibiting at least one modified movement based on at least two interference patterns in the time-sequence of interference patterns of the illuminated biological cell.

12. The device of claim 10, wherein the detector is configured to detect a biological cell exhibiting at least one modified movement based on partial holographic reconstructions of at least two interference patterns in the time-sequence of interference patterns of the illuminated biological cell.

13. The device of claim 10, wherein the tracker is configured to track the detected biological cell by illuminating the detected biological cell and tracking the interference pattern of the illuminated biological cell between successive images in the image series representing the time-sequence of interference patterns of the illuminated biological cell.

14. The device of claim 10, wherein the tracker is configured to track the detected biological cell by illuminating the detected biological cell and tracking a partial holographic reconstruction of the interference pattern of the illuminated biological cell between successive images in the image series representing the time-sequence of interference patterns of the illuminated biological cell.

15. The device of claim 10, wherein the light source is configured to emit at least partially coherent light.

16. The device of claim 1, wherein the actuator is configured to divert the detected biological cell within the flow of liquid in the flow channel by creating:
an electric field within the flow of liquid;
a jet flow within the flow of liquid by heating the liquid; or
a surface acoustic wave along an inner surface of the flow channel.

17. The device of claim 1, wherein:
at least one zone of the flow channel comprises at least one obstacle, thereby forming at least one zone of obstacles, the at least one obstacle having a lateral surface configured to obstruct a path of the biological cell carried by the flow of liquid, the lateral surface of the at least one obstacle of the at least one zone of obstacles being comprised within the least one CMM surface of the at least one zone of obstacles.

18. The device of claim 1, wherein the molecules coating at least one CMM surface of the at least one zone of the flow channel comprise at least one of: antibodies or aptamers.

19. The device of claim 1, wherein the at least one CMM surface of at least one zone of the flow channel is configured to modify the movement of the biological cell belonging to the associated cell category of the zone by binding and releasing the biological cell belonging to the associated cell category of the zone.

20. A method for sorting biological cells, the method comprising:
detecting a biological cell in a flow channel, wherein
the flow channel is configured to pass a flow of liquid, the flow of liquid carrying the biological cells to be sorted, the flow channel comprising at least one zone, each zone of the flow channel being associated with a cell category,
each zone of the flow channel comprises at least one surface coated with molecules having an affinity to the cell category associated with the zone, the at least one surface of the zone being configured to modify, by the molecules coating the at least one surface, a movement of a biological cell belonging to the associated cell category of the zone as the biological cell carried by the flow of liquid passes the zone, whereby the at least one surface forms at least one biological cell movement modifying (CMM) surface, and
the detected biological cell is a biological cell exhibiting at least one modified movement in one zone of the flow channel; and
communicating a trigger signal to an actuator configured to divert the detected biological cell within the flow of liquid in the flow channel based on the detection of the biological cell exhibiting at least one modified movement.
